(19) United States
(12) Patent Application Publication
(10) Pub. No.: US 2026/0199686 A1
(43) Pub. Date: Jul. 16, 2026

(54) CCM STIMULATION AS A TREATMENT FOR CARDIAC ANTI-INFLAMMATORY

(71) Applicant: Impulse Dynamics NV, Willemstad (CW)

(72) Inventors: Tamir BEN DAVID, Tel-Aviv (IL); David PRUTCHI, Voorhees, NJ (US)

(73) Assignee: Impulse Dynamics NV, Willemstad (CW)

(21) Appl. No.: 19/444,258

(22) Filed: Jan. 9, 2026

Related U.S. Application Data
(60) Provisional application No. 63/743,651, filed on Jan. 10, 2025.

(51) Int. Cl. A61N 1/365; A61N 1/362

(52) U.S. Cl. CPC A61N 1/36557; A61N 1/3627

(57) ABSTRACT

Systems and methods for suppressing a patient cardiac inflammation condition with cardiac contractility modulation stimulation therapy, the system comprising a implantable controller, the controller configured to carry out the method of: a. receiving at least one parameter indicative of an increase and/or a change in an inflammatory state in said patient; and b. providing cardiac contractility modulation stimulation therapy and suppressing a patient cardiac inflammation condition.

### RELATED APPLICATION

This application claims the benefit of priority of U.S. Provisional Patent Application No. 63/743,651 filed on Jan. 10, 2025, the contents of which are incorporated herein by reference in their entirety.

### FIELD AND BACKGROUND OF THE INVENTION

The systems and methods described herein include, among other things, systems and methods for treatment for cardiac inflammation levels and, more particularly, but not exclusively, to Cardiac Contractility Modulation stimulation for treatment for cardiac inflammation levels and to systems and methods for suppressing a patient cardiac inflammation condition with cardiac contractility modulation stimulation therapy.

Inflammation is a complex process that involves the activation of the immune system in response to injury or infection. In heart failure, inflammation is thought to play a role in the development and progression of the disease. There are several ways that inflammation can contribute to heart failure:

Inflammation can damage the heart muscle directly. This can happen through the release of harmful chemicals by immune cells, or through the activation of pro-inflammatory pathways.

Inflammation can also lead to cardiac remodeling that relates to a process that negatively affects the heart's structure and function. Remodeling may involve creation of scar tissue in the heart and abnormal growth of heart muscle cells.

Inflammation can impair the heart's ability to pump blood. This may happen by activation of pro-inflammatory pathways that may lead to vasoconstriction of the heart blood vessels, resulting with reduced blood flow to the body.

The level of inflammation in heart failure patients can be detected by measuring inflammatory markers such as C-reactive protein (CRP), Interleukin-1β (IL-1β), interleukin-6 (IL-6), Interleukin-18 (IL-18), Soluble ST2, ASC specks, Galectin-3, Caspase-1, Heart-type fatty acid binding protein (H-FABP) Tumor necrosis factor (TNF)-α and phospholipase lipoprotein A2.

There are several drugs for anti-inflammatory therapy in heart failure. These include drugs that inhibit the production or activity of pro-inflammatory cytokines, or that try to heal the inflammation. Inflammation may be present in patients with both heart failure with reduced ejection fraction (HFrEF) and with preserved ejection fraction (HFpEF). Treatment of inflammation may improve the symptoms and prevent the prognosis of heart failure condition.

Additional background art includes an article by D'Onofrio et al, titled “First human implant of the cardiac contractility modulation in patient with dilated cardiomyopathy-related laminopathy” (Heart Rhythm Case Reports 2023; 9:381-385) disclosing an improvement of quality of life, echo parameters, and inflammatory biomarkers following Cardiac Contractility Modulation device implantation. The results of the case report suggest that Cardiac Contractility Modulation could be a possible novel treatment in patients with dilated cardiomyopathy (DCM)-related laminopathy to prevent and/or delay the progression of heart failure.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.

Example 1. A system for suppressing a patient cardiac inflammation condition and/or an inflammation condition in a patient presenting a cardiac condition, comprising an implantable controller, said controller configured to carry out the method of:
a. receiving at least one parameter indicative of an increase and/or a change in an inflammatory state in said patient;
b. providing cardiac contractility modulation stimulation therapy and suppressing a patient cardiac inflammation condition.

Example 2. The system according to example 1, wherein said at least one parameter is a change and/or an increase in one or more inflammatory markers.

Example 3. The system according to example 1 or example 2, wherein said at least one parameter is a change and/or increase in a body temperature of said patient.

Example 4. The system according to any one of examples 1-3, wherein said at least one parameter is a change and/or an increase in a heart rate (HR) in said patient.

Example 5. The system according to any one of examples 1-4, wherein said at least one parameter is a change and/or a reduction in heart rate variability in said patient.

Example 6. The system according to any one of examples 1-5, wherein said at least one parameter is a low vagal tone.

Example 7. The system according to any one of examples 1-6, wherein said one or more inflammatory markers are markers for one or more of: Interleukin-6 and C-reactive protein (CRP).

Example 8. The system according to any one of examples 1-7, wherein said one or more inflammatory markers are markers for one or more of: phospholipase lipoprotein A2, Interleukin-1β (IL-1β), Interleukin-18 (IL-18), Soluble ST2, ASC specks, Galectin-3, Caspase-1, Heart-type fatty acid binding protein (H-FABP) and Tumor necrosis factor (TNF)-α levels.

Example 9. The system according to any one of examples 1-8, wherein said system further comprises at least one sensor configured for detecting said at least one parameter; and wherein said at least one parameter is received from said at least one sensor.

Example 10. The system according to any one of examples 1-9, wherein said at least one sensor is located within said system and implanted within a body of said patient.

Example 11. The system according to any one of examples 1-10, wherein said at least one sensor is located outside a body of said patient and wherein said indication is transmitted to said implantable controller.

Example 12. The system according to any one of examples 1-11, wherein said at least one parameter is provided to said implantable controller by an external source.

Example 13. The system according to any one of examples 1-12, wherein said external source is one or more of a physician, said patient and an external device.

Example 14. The system according to any one of examples 1-13, wherein said cardiac contractility modulation stimulation therapy comprises providing a pulse amplitude of 7.5V, a pulse width 5 ms and two bipolar pulses per cardiac cycle.

Example 15. The system according to any one of examples 1-14, wherein said providing comprises one or more of:
a. providing said effective amount of cardiac contractility modulation stimulation therapy for at least 5 hours of stimulation per day;
b. increasing a total of number of pulses provided per day an increment from about 5% to about 200%;
c. increasing a total of number of pulses provided per hour an increment from about 5% to about 200%;
d. increasing a pulse amplitude of pulses provided an increment from about 5% to about 200%;
e. increasing a pulse width of pulses provided an increment from about 5% to about 200%;
f. increasing a number of pulses provided per cardiac cycle an increment from about 5% to about 200%.

Example 16. The system according to any one of examples 1-15, wherein said increasing is performed by one or more of:
a. automatically by a cardiac contractility modulation stimulation device;
b. manually by said patient.

Example 17. The system according to any one of examples 1-16, wherein said controller is further configured to receive an indication of an improvement in said at least one parameter, and further configured for amending said effective amount of cardiac contractility modulation stimulation therapy accordingly.

Example 18. The system according to any one of examples 1-17, wherein said providing comprises providing an increased amount of said effective amount of cardiac contractility modulation stimulation therapy during late night and/or during early morning.

Example 19. The system according to any one of examples 1-18, wherein said providing comprises providing said effective amount of cardiac contractility modulation stimulation therapy during late night hours and/or during early morning hours.

Example 20. A method for suppressing a patient cardiac inflammation condition with cardiac contractility modulation stimulation therapy and/or an inflammation condition in a patient presenting a cardiac condition, the method comprising:
a. identifying at least one parameter indicative of an increase and/or a change in an inflammatory state in said patient;
b. providing cardiac contractility modulation stimulation therapy and suppressing a patient cardiac inflammation condition.

Example 21. The method according to example 20, wherein said at least one parameter is a change and/or an increase in one or more inflammatory markers.

Example 22. The method according to example 20 or example 21, wherein said at least one parameter is a change and/or increase in a body temperature of said patient.

Example 23. The method according to any one of examples 20-22, wherein said at least one parameter is a change and/or an increase in a heart rate (HR) in said patient.

Example 24. The method according to any one of examples 20-23, wherein said at least one parameter is a change and/or a reduce in heart rate variability in said patient.

Example 25. The method according to any one of examples 20-24, wherein said at least one parameter is a low vagal tone.

Example 26. The method according to any one of examples 20-25, wherein said one or more inflammatory markers are markers for one or more of: Interleukin-6 and C-reactive protein (CRP).

Example 27. The method according to any one of examples 20-26, wherein said one or more inflammatory markers are markers for one or more of: phospholipase lipoprotein A2, Interleukin-1β (IL-1B), Interleukin-18 (IL-18), Soluble ST2, ASC specks, Galectin-3, Caspase-1, Heart-type fatty acid binding protein (H-FABP) and Tumor necrosis factor (TNF)-α levels.

Example 28. The method according to any one of examples 20-27, wherein said identifying is performed by at least one sensor configured for detecting said at least one parameter; and wherein said at least one parameter is received from said at least one sensor.

Example 29. The method according to any one of examples 20-28, wherein said at least one sensor is located within a system and implanted within a body of said patient.

Example 30. The method according to any one of examples 20-29, wherein said at least one sensor is located outside a body of said patient and wherein said indication is transmitted to an implantable controller.

Example 31. The method according to any one of examples 20-30, wherein said at least one parameter is provided to an implantable controller by an external source.

Example 32. The method according to any one of examples 20-31, wherein said external source is one or more of a physician, said patient and an external device.

Example 33. The method according to any one of examples 20-32, wherein said effective amount of cardiac contractility modulation stimulation therapy comprises providing a pulse amplitude of 7.5V, a pulse width 5 mS and two bipolar pulses per cardiac cycle.

Example 34. The method according to any one of examples 20-33, wherein said providing comprises one or more of:
a. providing said effective amount of cardiac contractility modulation stimulation therapy for at least 5 hours of stimulation per day;
b. increasing a total of number of pulses provided per day an increment from about 5% to about 200%;
c. increasing a total of number of pulses provided per hour an increment from about 5% to about 200%;
d. increasing a pulse amplitude of pulses provided an increment from about 5% to about 200%;
e. increasing a pulse width of pulses provided an increment from about 5% to about 200%;
f. increasing a number of pulses provided per cardiac cycle an increment from about 5% to about 200%.

Example 35. The method according to any one of examples 20-34, wherein said increasing is performed by one or more of:
a. automatically by a cardiac contractility modulation stimulation device;
b. manually by said patient.

Example 36. The method according to any one of examples 20-35, further comprising receiving an indication of an improvement in said at least one parameter, and further comprising amending said effective amount of cardiac contractility modulation stimulation therapy accordingly.

Example 37. The method according to any one of examples 20-36, wherein said providing comprises providing an increased amount of said effective amount of cardiac contractility modulation stimulation therapy during late night and/or during early morning.

Example 38. The method according to any one of examples 20-37, wherein said providing comprises providing said effective amount of cardiac contractility modulation stimulation therapy during late night hours and/or during early morning hours.

Example 39. A method for suppressing a patient cardiac inflammation condition with cardiac contractility modulation stimulation therapy and/or an inflammation condition in a patient presenting a cardiac condition, the method comprising:
a. identifying at least one parameter indicative of an increase and/or a change in an inflammatory state in said patient;
b. providing cardiac contractility modulation stimulation therapy during late night hours and/or during early morning hours and suppressing a patient cardiac inflammation condition.

Example 40. The method according to example 39, wherein said at least one parameter is a change and/or an increase in one or more inflammatory markers.

Example 41. The method according to example 39 or example 40, wherein said at least one parameter is a change and/or increase in a body temperature of said patient.

Example 42. The method according to any one of examples 39-41, wherein said at least one parameter is a change and/or an increase in a heart rate (HR) in said patient.

Example 43. The method according to any one of examples 39-42, wherein said at least one parameter is a change and/or a reduce in heart rate variability in said patient.

Example 44. The method according to any one of examples 39-43, wherein said at least one parameter is a low vagal tone.

Example 45. The method according to any one of examples 39-44, wherein said one or more inflammatory markers are markers for one or more of: Interleukin-6 and C-reactive protein (CRP).

Example 46. The method according to any one of examples 39-45, wherein said one or more inflammatory markers are markers for one or more of: phospholipase lipoprotein A2, Interleukin-1β (IL-1β), Interleukin-18 (IL-18), Soluble ST2, ASC specks, Galectin-3, Caspase-1, Heart-type fatty acid binding protein (H-FABP) and Tumor necrosis factor (TNF)-α levels.

Example 47. The method according to any one of examples 39-46, wherein said identifying is performed by at least one sensor configured for detecting said at least one parameter; and wherein said at least one parameter is received from said at least one sensor.

Example 48. The method according to any one of examples 39-47, wherein said at least one sensor is located within a system and implanted within a body of said patient.

Example 49. The method according to any one of examples 39-48, wherein said at least one sensor is located outside a body of said patient and wherein said indication is transmitted to an implantable controller.

Example 50. The method according to any one of examples 39-49, wherein said at least one parameter is provided to an implantable controller by an external source.

Example 51. The method according to any one of examples 39-50, wherein said external source is one or more of a physician, said patient and an external device.

Example 52. The method according to any one of examples 39-51, wherein said effective amount of cardiac contractility modulation stimulation therapy comprises providing a pulse amplitude of 7.5V, a pulse width 5 mS and two bipolar pulses per cardiac cycle.

Example 53. The method according to any one of examples 39-52, wherein said providing comprises one or more of:
a. providing said effective amount of cardiac contractility modulation stimulation therapy for at least 5 hours of stimulation per day;
b. increasing a total of number of pulses provided per day an increment from about 5% to about 200%;
c. increasing a total of number of pulses provided per hour an increment from about 5% to about 200%;
d. increasing a pulse amplitude of pulses provided an increment from about 5% to about 200%;
e. increasing a pulse width of pulses provided an increment from about 5% to about 200%;
f. increasing a number of pulses provided per cardiac cycle an increment from about 5% to about 200%.

Example 54. The method according to any one of examples 39-53, wherein said increasing is performed by one or more of:
a. automatically by a cardiac contractility modulation stimulation device;
b. manually by said patient.

Example 55. The method according to any one of examples 39-54, further comprising receiving an indication of an improvement in said at least one parameter, and further comprising amending said effective amount of cardiac contractility modulation stimulation therapy accordingly.

Example 56. The method according to any one of examples 39-55, wherein said providing comprises providing an increased amount of said effective amount of cardiac contractility modulation stimulation therapy during late night and/or during early morning.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the systems and methods described herein may be embodied as a system, method or computer program product. Accordingly, some embodiments of the systems and methods described herein may take the form of an entirely hardware embodiment, an entirely software embodiment (including firmware, resident software, micro-code, etc.) or an embodiment combining software and hardware aspects that may all generally be referred to herein as a “circuit,” “module” or “system.”

### BRIEF DESCRIPTION OF THE SEVERAL VIEWS OF THE DRAWINGS

Some embodiments of the invention are herein described, by way of example only, with reference to the accompanying drawings.

In the drawings:

FIG. 1a is a block diagram of an exemplary cardiac device that includes one or more leads, which are couplable to device at one or more can connectors, according to some embodiments of the invention;

FIG. 1b is a schematic diagram of an exemplary implantable cardiac device, according to some embodiments of the invention;

FIG. 1c is a schematic diagram of an exemplary implantable cardiac device configured to function also as cardioverter defibrillator (ICD), according to some embodiments of the invention;

FIGS. 2a-2d, are schematic representations of exemplary locations for positioning leads, according to some embodiments of the invention;

FIG. 3a is a schematic representation of exemplary monophasic pulses or biphasic pulses of Cardiac Contractility Modulation stimulation, according to some embodiments of the invention;

FIG. 3b is a schematic representation of an exemplary pulse train, according to some embodiments of the invention;

FIG. 4 is a flowchart of an exemplary method to suppress patient cardiac inflammation condition and/or an inflammation condition in a patient presenting a cardiac condition, according to some embodiments of the invention;

FIG. 5 is a flowchart of an exemplary method to suppress patient cardiac inflammation condition and/or an inflammation condition in a patient presenting a cardiac condition, according to some embodiments of the invention;

FIG. 6 is a flowchart of an exemplary method to suppress patient cardiac inflammation condition and/or an inflammation condition in a patient presenting a cardiac condition, according to some embodiments of the invention;

FIG. 7 is a flowchart of an exemplary method to suppress patient cardiac inflammation condition and/or an inflammation condition in a patient presenting a cardiac condition, according to some embodiments of the invention;

FIG. 8 is a flowchart of an exemplary method to suppress patient cardiac inflammation condition and/or an inflammation condition in a patient presenting a cardiac condition, according to some embodiments of the invention; and

FIG. 9 is a flowchart of an exemplary method to suppress patient cardiac inflammation condition and/or an inflammation condition in a patient presenting a cardiac condition, according to some embodiments of the invention.

### DESCRIPTION OF SPECIFIC EMBODIMENTS OF THE INVENTION

The systems and methods described herein, in some embodiments, relate to systems and methods for treatment of cardiac inflammation and, more particularly, but not exclusively, to Cardiac Contractility Modulation stimulation for treatment of cardiac inflammation.

### Overview

The systems and methods described herein apply CCM to provide therapeutic treatment for cardiomyopathy related to inflammation of the heart. In one actual example, the systems and methods described herein provided treatment for a 48-year-old white man, suffering from chronic heart failure with reduced ejection fraction and who was screened for dilated cardiomyopathy (DCM)-related laminopathy (LMNA). As set forth in the example data below, therapy scheduled and applied for 8 hours CCM stimulation per day reduced and treated the inflammation of the patient's heart.

Specifically, a 48-year-old white man, suffering from chronic heart failure with reduced ejection fraction, was screened for dilated cardiomyopathy (DCM)-related laminopathy (LMNA). Therapy was scheduled for 8 hours CCM stimulation per day.

At 6 and 12 months follow-up, the patient showed improvements in clinical symptoms, quality of life, laboratory biomarkers, and left ventricular systolic function parameters on transthoracic echocardiogram, as exhibited in the following table:

| | | | | Normal |
|---|---|---|---|---|
| Parameter | Baseline | 6 months | 12 months | Values |
| KCCQ | 75.5 | 87.3 | 93.6 | 75-100 |
| LVEDV [mL] | 133 | 81 | 125 | 106 ± 22 |
| LVESV [mL] | 80 | 50 | 64 | 41 ± 10 |
| LVEF [%] | 40 | 50 | 50 | 62 ± 5 |
| GLS [%] | −11.3 | −13 | −12.8 | −18 ± 2 |
| RVD1 [mm] | 46 | 39 | 39 | 33 ± 4 |
| RVD2 [mm] | 42 | 42 | 42 | 27 ± 4 |
| RVD3 [mm] | 75 | 69 | 69 | 71 ± 6 |
| NT-proBNP [pg/mL] | 1520 | 801 | 675 | 0-125 |
| Laminin [ng/mL] | 34 | 28.13 | 13 | 0-50 |
| Collagen 4 [ng/mL] | 33 | 15.6 | 21 | 0-30 |
| Collagen 3 [ng/mL] | 18.1 | 16.5 | 21.3 | 0-30 |
| Copeptin [pmol/L] | 17.54 | 14.92 | 11.32 | <17 |
| C-cystatin [mg/L] | 1.14 | 1.01 | 1.12 | 0.47-1.09 |
| Lp-PLA2 [ng/mL] | 1628 | 26.57 | 201.5 | 1-200 |
| Interleukin-6 [pg/mL] | 5.1 | 3.6 | <2 | 0-5 |

GLS 5 = global longitudinal strain; KCCQ = Kansas City Cardiomyopathy Questionnaire.
Clear improvement was seen in the following inflammatory markers: Interleukin-6 (IL-6) and phospholipase lipoprotein A2.

An aspect of some embodiments of the systems and methods described herein relates to providing cardiac contractility modulation treatment, optionally with an implantable pulse generator (IPG) that provides cardiac contractility modulation treatment, to suppress patient cardiac inflammation condition and/or an inflammation condition in patients having also cardiac conditions (for example heart failure). In some embodiments, the patient already has implanted an IPG, which is configured to treat a cardiac condition and/or configured to provide cardiac contractility modulation treatment. Typically, the IPG devices described herein apply electrical stimulation to treat the cardiac condition and/or the inflammation condition in the patient. To this end, these typical embodiments are IPG devices that include a programmable controller that stores parameters where at least one parameter is indicative of an increase and/or a change in an inflammatory state is identified in the patient, and then the controller will initiate cardiac contractility modulation treatment.

In some examples, the cardiac contractility modulation treatment is provided to reduce level of Interleukin-6 (IL-6) and/or phospholipase lipoprotein A2. In other embodiments, the cardiac contractility modulation treatment is provided to reduce a level of one or more C-reactive protein (CRP), Interleukin-1β (IL-1β), Interleukin-18 (IL-18), Soluble ST2, ASC specks, Galectin-3, Caspase-1, Heart-type fatty acid binding protein (H-FABP) Tumor necrosis factor (TNF)-α and phospholipase lipoprotein A2.

In some embodiments, the devices described herein provide cardiac contractility modulation treatment in an amount sufficient to increase vagal tone and by that activate the vagal anti-inflammatory pathway. In one particular embodiment, the cardiac contractility modulation treatment is provided in concomitance with close loop monitoring. This close loop monitoring typically comprises periodically measuring inflammatory markers using blood tests. Other markers, besides inflammatory markers, may be monitored as well and the device may be used with a monitoring process that periodically measures changes of temperature in the patient, optionally an increase in temperature over a predetermined threshold and optionally periodically measures changes in heart rate and/or heart rate variability. Measurements from the monitoring may be used to determine the cardiac contractility modulation treatment to provide. Cardiac contractility modulation treatment stimulation may be delivered at a predetermined total amount of cardiac contractility modulation per day, for example starting with 5 to 8 hours per day. For example, the effect of the cardiac contractility modulation treatment stimulation on inflammation markers is measured to assess the therapeutic effects.

In some embodiments, the cardiac contractility modulation treatment stimulation parameters (including number of hours, number of pulses per cardiac cycle, pulse amplitude) are tuned in order to maximize the effect on reducing the level of the inflammation markers. In some embodiments, the systems and methods described herein provide the cardiac contractility modulation stimulation during late night and early morning time (for example, between 20:00 and midnight and/or between 02:00 and 04:00), which are the times when inflammation is exacerbated and symptoms and mortality rates are highest. In some embodiments, the therapeutic effects are seen after a single treatment. In some embodiments, the therapeutic effects are seen after two or more treatments. In some embodiments, more cardiac contractility modulation stimulation is provided when body temperature is elevated above set temperature threshold. In some embodiments, more cardiac contractility modulation stimulation is provided when heart rate is within set range (preferably above and/or below an average predetermined HR). In some embodiments, cardiac contractility modulation stimulation is provided when heart rate variability is reduced below a set threshold since there is an inverse relationship between the levels of HRV and inflammatory markers like CRP and/or IL-6. In some embodiments, the reduction in the level of the inflammation markers is from about 50% to about 80%, optionally from about 40% to about 90%, optionally from about 30% to about 100%.

Therefore, as mentioned above, in some embodiments, cardiac contractility modulation stimulation is provided in one or more of the following scenarios: when body temperature is elevated above set temperature threshold; when heart rate is within set range (preferably above average HR); and when heart rate variability is reduced below a set threshold. In some embodiments, the patient is monitored using one or more of the following sensors: body temperature sensor, heart rate (HR) sensor, oxygen saturation sensor, sleep sensor, respiration sensor, movement sensor. The parameters that are being monitored are one of more of: inflammatory markers, body temperature, heart rate variability (HRV), HR and oxygen saturation. Inflammatory markers are monitored using for example blood tests or any other current technology that allows (optionally, real-time) monitoring of inflammatory markers.

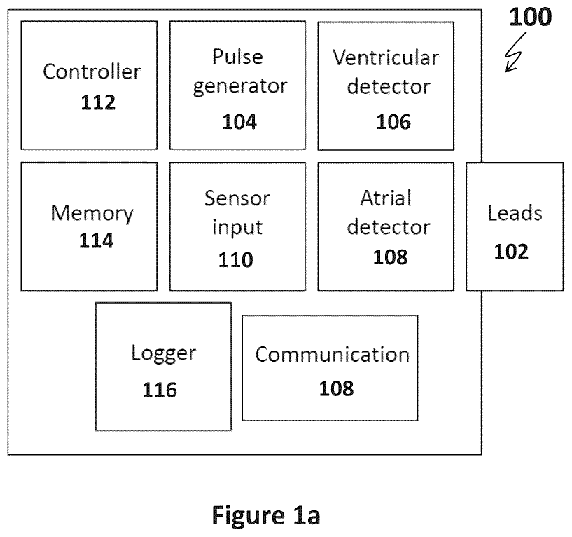

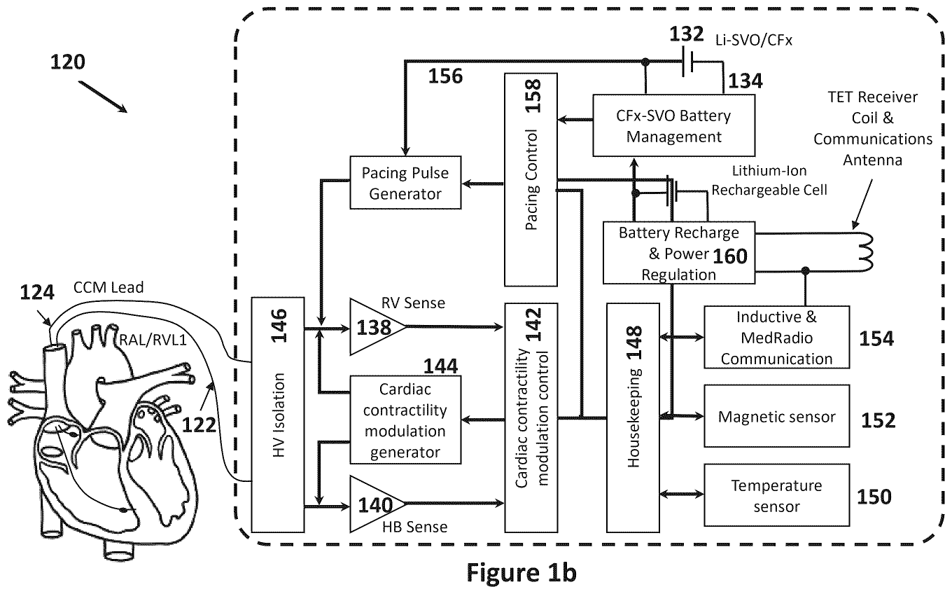

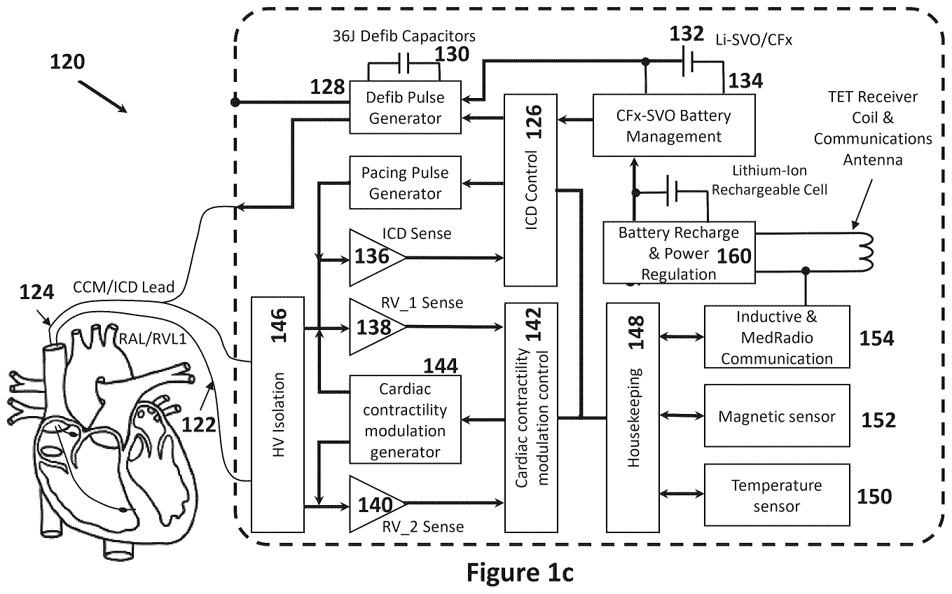

FIG. 1a depicts a block diagram of an exemplary cardiac device 100 that includes one or more leads 102 (optionally two leads), which are couplable to device 100 at one or more can connectors (not shown). In some embodiments, one lead is connected in a location in either the right atrium (RA) or in the right ventricle (RV), while another lead (or more than one lead) is connected at a location in the right ventricle. In the following explanations, the lead connected to the location in the right atrium will be referred to RA lead or RAL, the first lead connected to the location in the right ventricle will be referred to RV1 lead or RVL1 while the second lead connected at a location in the right ventricle will be referred to RV2 lead or RVL2.

The depicted pulse generator 104 generates the signal that stimulates the patient's heart. The pulse generator 104 includes power circuitry, for example, one or more storage capacitors of the type used for generating a signal for stimulating the heart of a patient.

Optionally, a ventricular detector 106 is provided and used to detect atypical ventricular activation, which can be a contra-indication to signal application. The device 100 includes an atrial detector 108 that detects atypical atrial activation, which may be used as an input to decision making by device 100.

A sensor input 110 receives data from one or more sensors, typically electrical sensors but they may be any suitable sensor, such as flow, temperature, pressure and/or acceleration sensors, body as well as temperature sensors, heart rate (HR) sensors, oxygen saturation sensors, sleep sensors, respiration sensors, and/or movement sensors. Data from the sensors are processed (e.g., by the depicted controller 112 and/or detectors 106, 108) and are used as an input to decision making processes in device 100.

The controller 112 is illustrated as part of the depicted device 100 and the controller 112 executes one or more logics to control operation of the device 100, for example, controlling a timing and/or other parameters of a signal and/or if a signal is to be applied.

The controller 112 of the depicted device 100 controls the application of stimulation pulses according to a treatment plan. Optionally, the controller 112 effects a change in the plan, for example so as to compensate for real time deviations from the treatment plan (e.g., a skipped stimulation).

A depicted memory 114 is provided to store logic, past effects, therapeutic plan, adverse events, and/or pulse parameters.

In some embodiments, the controller 112 and/or memory 114 are programmed with one or more treatment plans (optionally set for the specific patient) and/or with one or more fallback treatment plans.

In some embodiments, instructions for “compensating” for a change from the planned treatment are stored and are addressed by the device controller when relevant, including for example modifications in treatment duration, number of stimulations, stimulation signal parameters (e.g., current intensity), and/or other modifications which may be applied to compensate for a real time change in the original plan. In some embodiments, the instructions include numerical factors according to which one or more parameters of treatment are modified. In some examples, the instructions may include: a factor by which the stimulation current intensity should be multiplied in case the actual applied stimulations did not reach the target amount of energy; a factor by which output voltage should be modulated to result in the desired current intensity; a factor by which the stimulation rate should be multiplied in case the actual applied stimulation did not reach the target total number of stimulations (optionally within a set time period); time related modifications such as factors by which the treatment session duration should be lengthened in case a target was not reached; and the like.

In some embodiments, the controller refers to a look-up table or the like which ties between specific situations (e.g., a skipped stimulation, number of actual stimulations delivered being lower than planned, etc.) and the instructions for compensating for that situation (e.g., by updating one or more parameters, such as updating the length of a treatment session).

A logger 116 is optionally provided to store activities of device 100 and/or of the patient. Such a log and/or programming may use a communication module 118 (e.g., of a type known in the art) to send data from device 100, for example, to a programmer (not shown) and/or to receive data, for example, programming, for example, pulse parameters.

FIG. 1b, shows a schematic diagram of an exemplary implantable cardiac device 120, according to some embodiments of the invention.

The depicted device 120 is configured to perform a plurality of actions, for example, one or more of:
1. Configured for delivery of cardiac electrical stimulation, such as Cardiac Contractility Modulation stimulation.
2. Configured to function as a pacing device, as a Right Atrium Pacing (RAP) device that performs Right Atrium Pacing (RAP) or a Right Ventricle Pacing (RVP) device that performs Right Ventricle Pacing (RVP).

In some embodiments, the device comprises an RAP/RVP lead 122 (also referred as Right Atrium lead or RAL, or to Right Ventricle lead 1 or RVL1), and a Cardiac Contractility Modulation lead 124.

In some embodiments, the device comprises a power source (e.g., a battery 132) and power source management circuitry 134. In some embodiments, the device optionally comprises a battery recharge and power regulation unit 160 configured for configured for recharging the rechargeable battery (when one is used) and for regulating the power between batteries. In some embodiments, the device comprises a pacing pulse generator 156 that is controlled by a pacing control unit 158. In some embodiments, activation of the one or more cardiac contractility modulation lead(s) 124, which are optionally positioned in the right ventricle (RVL2), is by a cardiac contractility modulation module which includes or is connected to: Cardiac Contractility Modulation control 142, a Cardiac Contractility Modulation generator 144.

In some embodiments, the leads are connected to isolation 146, which are also passed through one or more (two are shown) RV sense 138, 140.

In some embodiments, the device comprises a housekeeping module 148, which includes or is connected to one or more sensor such as a temperature sensor 150, a magnetic sensor 152, and communication means 154 such as an antenna, a receiver and the like. Other sensors may include flow sensors, pressure sensors, acceleration sensors, heart rate (HR) sensor, oxygen saturation sensor, sleep sensor, respiration sensor, movement sensor and/or other transducers.

In some embodiments, data received from the one or more sensors are received as input. Optionally, the input is processed by the device control (e.g., by the cardiac contractility modulation control, and/or a general controller, not shown) and is optionally used as input to decision making processes in device 120.

In some embodiments, the device control (e.g., the Cardiac Contractility Modulation control, and/or a general controller, not shown) executes one or more logics to decide, for example, a timing and/or other parameters of a signal and/or if a signal is to be applied.

A memory (not shown) is optionally provided, for example, to store logic, past effects, therapeutic plan, adverse events and/or pulse parameters.

A logger (not shown) is optionally provided to store activities of device 120 and/or of the patient. Such a log and/or programming may use a communication module 154 to send data from device 120, for example, to a programmer (not shown) and/or to receive data, for example, programming, for example, pulse parameters.

Referring now to FIG. 1c, showing a schematic diagram of an exemplary implantable cardiac device 120 configured to function also as cardioverter defibrillator (ICD), according to some embodiments of the invention.

In some embodiments, the device is configured for performing a plurality of actions, for example, one or more of:
1. Configured for delivery of cardiac electrical stimulation, such as Cardiac Contractility Modulation stimulation.
2. Configured to function as cardioverter defibrillator (ICD).
3. Configured to function as a pacing device, as a Right Atrium Pacing (RAP) device that performs Right Atrium Pacing (RAP) or a Right Ventricle Pacing (RVP) device that performs Right Ventricle Pacing (RVP).

In some embodiments, the device comprises an RAP/RVP lead 122 (also referred as Right Atrium lead or RAL, or to Right Ventricle lead 1 or RVL1), and a Cardiac Contractility Modulation lead 124. In some embodiments, the Cardiac Contractility Modulation lead 124 is also used as an ICD lead.

In some embodiments, activation of the ICD lead is by an ICD module which includes or is connected to: ICD control 126, a defibrillation pulse generator 128 (via one or more capacitors 130), a power source (e.g., a battery 132) and power source management circuitry 134, and ICD sense 136 which senses an applied pulse to verify the pulse is within a selected (e.g., programmed) amplitude and/or duration. In some embodiments, the device optionally comprises a battery recharge and power regulation unit 160 configured for recharging the rechargeable battery (when one is used) and for regulating the power between batteries. In some embodiments, the device comprises a pacing pulse generator 156 that is controlled by a pacing control unit (not shown). In some embodiments, activation of the one or more cardiac contractility modulation lead(s) 124, which are optionally positioned in the right ventricle (RVL2), is by a cardiac contractility modulation module which includes or is connected to: Cardiac Contractility Modulation control 142, a Cardiac Contractility Modulation generator 144.

It is noted that in some embodiments, the ICD coil and one or more electrodes for pacing and/or cardiac contractility modulation are configured on the same lead.

In some embodiments, the leads are connected to isolation 146, which are also passed through one or more (two are shown) RV sense 138, 140.

In some embodiments, the device comprises a housekeeping module 148, which includes or is connected to one or more sensor such as a temperature sensor 150, a magnetic sensor 152, and communication means 154 such as an antenna, a receiver and the like. Other sensors may include flow sensors, pressure sensors, acceleration sensors, heart rate (HR) sensor, oxygen saturation sensor, sleep sensor, respiration sensor, movement sensor and/or other transducers.

In some embodiments, data received from the one or more sensors are received as input. Optionally, the input is processed by the device control (e.g., by the ICD control, cardiac contractility modulation control, and/or a general controller, not shown) and is optionally used as input to decision making processes in device 120.

In some embodiments, the device control (e.g., the ICD control, Cardiac Contractility Modulation control, and/or a general controller, not shown) executes one or more logics to decide, for example, a timing and/or other parameters of a signal and/or if a signal is to be applied.

A memory (not shown) is optionally provided, for example, to store logic, past effects, therapeutic plan, adverse events and/or pulse parameters.

A logger (not shown) is optionally provided to store activities of device 120 and/or of the patient. Such a log and/or programming may use a communication module 154 to send data from device 120, for example, to a programmer (not shown) and/or to receive data, for example, programming, for example, pulse parameters.

### Exemplary Positioning of Leads

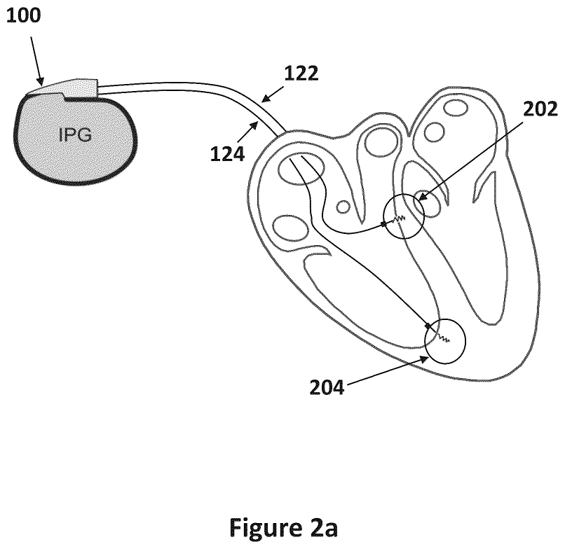

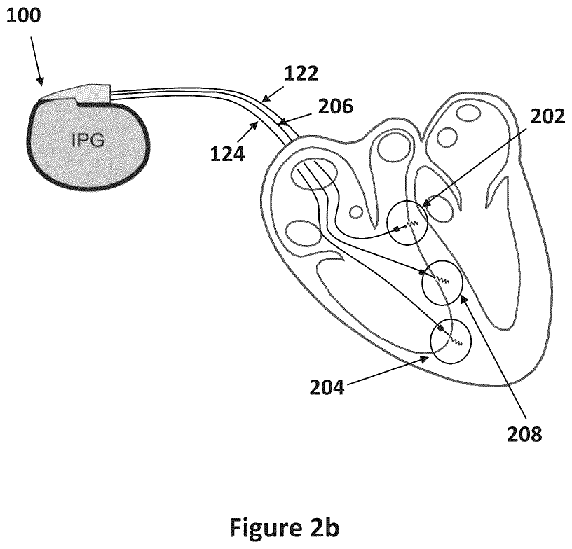

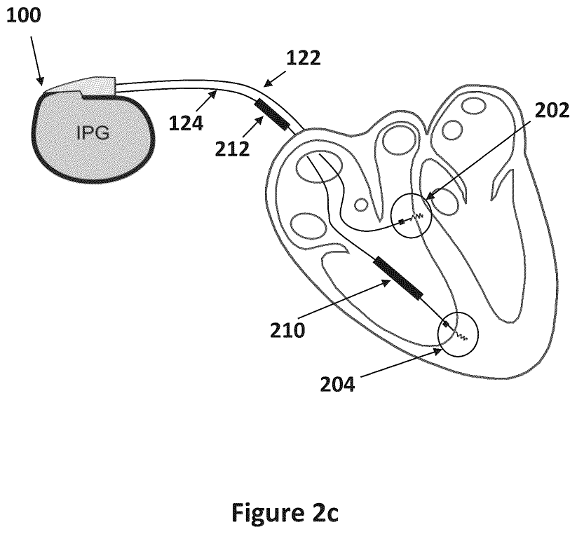

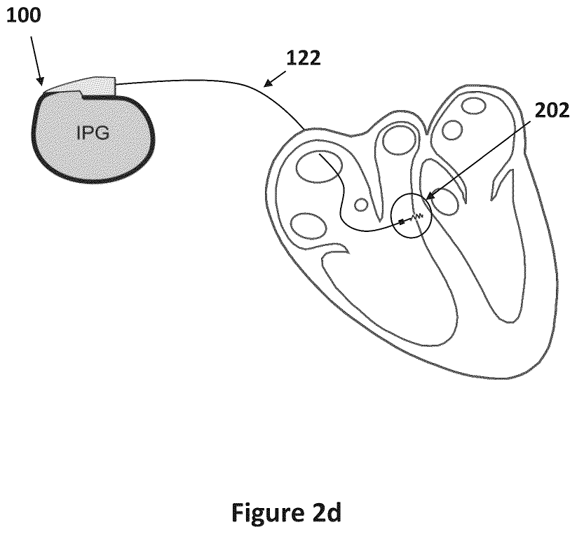

Referring now to FIGS. 2a-2d, showing schematic representation of exemplary locations for positioning leads, according to some embodiments of the invention. Same parts are provided with same reference numbers.

In some embodiments, one or more leads are positioned within the heart of the patient.

In some embodiments, an exemplary cardiac therapy device 100 (or implantable pulse generator-IPG) comprises 2 leads, as shown for example in FIG. 2a:
1. An RA lead 122 connected to a location within the heart in the right atrium 202; and
2. A Cardiac Contractility Modulation lead 124, optionally connected to a location 204 within the right ventricle (RVL2).

In some embodiments, the RVL2 is used for providing Cardiac Contractility Modulation and/or pacing.

In some embodiments, the RAL is used for pacing and optionally also for Cardiac Contractility Modulation.

In some embodiments, Cardiac Contractility Modulation treatment is provided by any of the leads. In some embodiments, Cardiac Contractility Modulation treatment can be provided by one or more of the leads in more than one fashion, for example in one or more of the following fashions:
1. Only one lead provides Cardiac Contractility Modulation treatment at a certain time;
2. Cardiac Contractility Modulation treatment is provided simultaneously via the two leads;
3. Cardiac Contractility Modulation treatment is provided sequentially via the two leads.

In some embodiments, an exemplary cardiac therapy device 100 (or implantable pulse generator-IPG) comprises 3 leads, as shown for example in FIG. 2b:
1. An optional RAL 122 connected to a location within the right atrium 202; and
2. Two RVLs 124 and 206 (RVL1 and RVL2), connected to two locations 204 and 208 within the right ventricle, for example located in the RV septum and Apex.

In some embodiments, either RVL (1 or 2) is used for providing Cardiac Contractility Modulation and/or pacing.

In some embodiments, the optional RAL is used for pacing and optionally also for Cardiac Contractility Modulation.

In some embodiments, Cardiac Contractility Modulation treatment is provided by any of the leads. In some embodiments, Cardiac Contractility Modulation treatment can be provided by one or more of the leads in more than one fashion, for example in one or more of the following fashions:
1. Only one lead provides Cardiac Contractility Modulation treatment at a certain time;
2. Cardiac Contractility Modulation treatment is provided simultaneously via two leads or three leads;
3. Cardiac Contractility Modulation treatment is provided sequentially via two leads or three leads.

In some embodiments, an exemplary cardiac therapy device 100 (or implantable pulse generator—IPG) comprises 2 leads, as shown for example in FIG. 2c:
1. An RAL connected to a location within the heart in the right atrium 202 (or a right ventricle lead RVL1—not shown); and
2. An RVL 124, connected to a location 204 within the right ventricle.

In some embodiments, the RAL is used for pacing and optionally also for Cardiac Contractility Modulation (RVL2).

In some embodiments, the RVL2 is used for providing Cardiac Contractility Modulation and/or pacing and/or defibrillation. In some embodiments, the RVL further comprises a single RV coil 210, or dual RV coils having one RV coil 210 within the RV and an additional RV coil 212 in the Superior Vena Cava (SVC).

In some embodiments, Cardiac Contractility Modulation treatment is provided by any of the leads. In some embodiments, Cardiac Contractility Modulation treatment can be provided by one or more of the leads in more than one fashion, for example in one or more of the following fashions:
1. Only one lead provides Cardiac Contractility Modulation treatment at a certain time;
2. Cardiac Contractility Modulation treatment is provided simultaneously via the two leads;
3. Cardiac Contractility Modulation treatment is provided sequentially via the two leads.

In some embodiments, an exemplary cardiac therapy device 100 (or implantable pulse generator-IPG) comprises 1 lead, as shown for example in FIG. 2d:
1. An RAL connected to a location within the heart in the right atrium 202.

In some embodiments, the RAL is used for pacing and also for Cardiac Contractility Modulation.

### Exemplary Activation of the Exemplary Cardiac Therapy Device 100

The exemplary cardiac therapy device is configured for providing pacing treatment and/or Cardiac Contractility Modulation treatment and/or optionally defibrillation treatment, while the pacing is provided through the right atrium and the Cardiac Contractility Modulation treatment is provided at the right atrium and/or at a different location, for example at the right ventricle (RV). Cardiac Contractility Modulation treatment is provided to treat cardiac conditions and/or inflammation conditions.

### Exemplary Pacing

In some embodiments, the exemplary cardiac therapy device is configured for providing “physiological” cardiac pacing, which includes prompt activation of the left ventricle. In some embodiments, cardiac pacing is done by providing one or more pacing pulses to the RAL. In some embodiments, cardiac pacing can be done by providing pacing pulses to both RAL/RVL1 and RVL2. In some embodiments, when providing pacing pulses to both RAL/RVL1 and RVL2, the providing comprises a delay between the timing of the pacing pulse in the RAL/RVL1 and in the RVL2.

### Exemplary Cardiac Contractility Modulation Stimulation

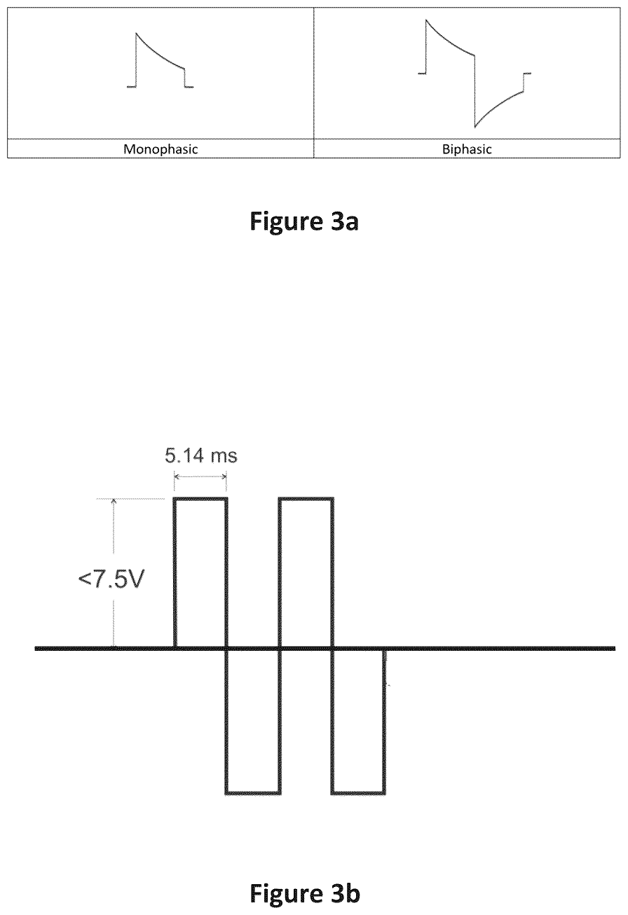

In some embodiments, Cardiac Contractility Modulation stimulation is delivered through the RAL/RVL1, the RVL2 or both. In some embodiments, when providing Cardiac Contractility Modulation stimulation via both RAL/RVL1 and RVL2, the providing comprises a delay between the timing of the Cardiac Contractility Modulation pulse in the RAL/RVL1 and in the RVL2. In some embodiments, Cardiac Contractility Modulation stimulation is delivered following delivery of a pacing pulse within the same cardiac cycle. In some embodiments, the Cardiac Contractility Modulation stimulation is delivered after a time delay (typically 5-75 ms) from the provision of the pacing pulse. In some embodiments, optionally, a detection of the R-wave is performed before the delay. In some embodiments, the Cardiac Contractility Modulation stimulation pulse is used for the HBP. In some embodiments, Cardiac Contractility Modulation stimulation comprises monophasic pulses or biphasic pulses, as shown for example in FIG. 3a. In some embodiments, the Cardiac Contractility Modulation consists of a pulse train of 2 to 4 biphasic pulses, where each biphasic pulse has a duration of 10 to 14 ms, and a pulse amplitude of ±7.5V, as shown for example in FIG. 3b. In some embodiments, the Cardiac Contractility Modulation stimulation parameters are a biphasic pulse amplitude of from about ±1V to about ±9V, a biphasic pulse width of from about 4 ms to about 10 ms duration per phase, and from 1 to 4 biphasic pulses per cardiac cycle, optionally at a time that the ventricle is in its absolute refractory period (e.g., 20 to 50 ms after the start of electrical depolarization).

### Exemplary General Features

In some embodiments, the RAL/RVL1 provides pacing while the RVL2 provides Cardiac Contractility Modulation. In some embodiments, the RVL2 provides pacing and the RAL/RVL1 provides Cardiac Contractility Modulation. In some embodiments, a controller comprises instructions to assess which lead provides pacing and which lead provides Cardiac Contractility Modulation, or both. In some embodiments, one or more of the RAL/RVL1 and RVL2 are used to measure intra cardiac electrogram signals. In some embodiments, by comparing the intra cardiac electrogram signals, the system identifies if there is a block in an area of the heart. In some embodiments, if a patient is found to suffer from a block, then the system provides dual pacing of RAL/RVL1 and RVL2. In some embodiments, if a patient is found to suffer from a block, a lead is placed in the RV septum.

### Exemplary Methods

In some embodiments, the implantable pulse generator 100 provides cardiac contractility modulation treatment in order to suppress patient cardiac inflammation condition and/or provides cardiac contractility modulation treatment to treat inflammation conditions in patients having cardiac conditions.

In some embodiments, more specifically, the cardiac contractility modulation treatment is provided in order to reduce level of Interleukin-6 (IL-6) and phospholipase lipoprotein A2.

In some embodiments, the cardiac contractility modulation treatment is provided in order to reduce level of one or more of C-reactive protein (CRP), Interleukin-1β (IL-1β), Interleukin-18 (IL-18), Soluble ST2, ASC specks, Galectin-3, Caspase-1, Heart-type fatty acid binding protein (H-FABP) Tumor necrosis factor (TNF)-α and phospholipase lipoprotein A2.

In some embodiments, the cardiac contractility modulation treatment is provided in order to increase vagal tone and by that activate the vagal anti-inflammatory pathway.

In some embodiments, the cardiac contractility modulation treatment is provided in concomitance with close loop monitoring.

In some embodiments, monitoring comprises periodically measuring one or more of inflammatory markers, body temperature, heart rate variability (HRV), HR and oxygen saturation. In some embodiments, inflammatory markers are preferably monitored using blood tests.

In some embodiments, providing the cardiac contractility modulation treatment stimulation comprises providing at set dose, for example starting with 8 hours per day.

In some embodiments, as part of the method of treating, measurements of the effect of the cardiac contractility modulation treatment stimulation on inflammation markers are assessed.

In some embodiments, the cardiac contractility modulation treatment stimulation parameters (including number of hours, number of pulses per cardiac cycle, pulse amplitude) are tuned and/or amended in order to maximize the effect on reducing the level of the inflammation markers.

In some embodiments, the cardiac contractility modulation stimulation is provided during late night and early morning time (as mentioned above), which are the times when inflammation is usually exacerbated and symptoms and mortality rates are highest.

In some embodiments, more cardiac contractility modulation stimulation is provided when body temperature is elevated above set temperature threshold. For example, more than 37.5 degrees Celsius (for example, 38, 38.5, 39 degrees Celsius), and/or more than 0.5 degrees Celsius (for example, 1, 1.5, 2 degrees Celsius) above a pre-measured normal patient body temperature (can be for example an hourly, daily, weekly, while moving-average measurement), and/or more than 0.5 degrees Celsius (for example, 1, 1.5, 2 degrees Celsius) above the patient body temperature at that specific time of day. Such value is measured before (can be for example an hourly, daily, weekly, while moving-average measurement).

In some embodiments, more cardiac contractility modulation stimulation is provided when heart rate is within set range (preferably above average HR). For example, more than 5 beats per minute (BPM) (for example, 10, 15, 20 BPM) above the normal patient HR that was previously (can be for example an hourly, daily, weekly, while moving-average measurement), and/or more than 5 beats per minute (BPM) (for example, 10, 15, 20 BPM) above the patient HR at that specific time of day (can be for example an hourly, daily, weekly, while moving-average measurement).

In some embodiments, cardiac contractility modulation stimulation is provided when heart rate variability is reduced below a set threshold since there is an inverse relationship between the levels of HRV and inflammatory markers like CRP and IL-6. For example, HRV measured over a period of 1 minute, HRV below 35 mS (for example, 30, 25, 20 mS), and/or HRV Less than 10 mS (for example, 15, 20, 25 mS) below the normal patient HRV that was previously measured (can be for example an hourly, daily, weekly, while moving-average measurement), and/or less than 10 mS (for example, 15, 20, 25 mS) below the normal patient HRV that was measured at that specific time of day (can be for example an hourly, daily, weekly, while moving-average measurement).

For IL-6, for example, a IL-6 level above 5 μg/mL (for example, 10, 15, 20 μg/mL), and/or IL-6 increase level by 5 μg/mL (for example, 10, 15, 20 μg/mL) above the normal patient Il-6 level that was previously measured.

In some embodiments, the already implanted device comprises a controller configured to receive one or more indications, and the implanted device is used to provide cardiac contractility modulation stimulation therapy in one of more cases according to the indications received. In some embodiments, the indications are received by one or more sources, for example by one or more sensors located within the implanted device, by one or more sensors located outside the device, positioned on the body of the patient and configured to transmit data to the controller of the implanted device, and from an external electronic device and/or server. In some embodiments, exemplary sensors area one or more of body temperature sensor, heart rate (HR) sensor, oxygen saturation sensor, sleep sensor, respiration sensor, movement sensor. In some embodiments, a dedicated personnel, for example a physician, provides the indication manually using the one or more external electronic devices. In some embodiments, the patient is allowed to begin the activation of the cardiac contractility modulation stimulation therapy manually. In some embodiments, the doctor begins the activation of the cardiac contractility modulation stimulation therapy manually. In some embodiments, a sensor in the device is automatically activated when certain parameters are reached (see below).

### Exemplary Events Triggering Acute Non-Excitatory Electrical Heart Failure Therapy

In some embodiments, cardiac contractility modulation stimulation therapy is provided to a patient when one or more parameters indicative of an increase and/or a change in an inflammatory state are received by the system. For example, when one or more of the following events happens and/or after receiving an indication that one or more of the following events are/had occurred:
1. An increase in Interleukin-6 (IL-6) has been identified;
2. An increase in phospholipase lipoprotein A2 has been identified;
3. An increase in one or more of C-reactive protein (CRP), Interleukin-1β (IL-1β), Interleukin-18 (IL-18), Soluble ST2, ASC specks, Galectin-3, Caspase-1, Heart-type fatty acid binding protein (H-FABP) Tumor necrosis factor (TNF)-α has been identified;
4. When a low vagal tone in a patient is found or an increase in vagal tone is desired; and
5. When an increase in temperature is detected, optionally above a certain threshold.

In some embodiments, the patient already has implanted a device configured to provide cardiac contractility modulation therapy for a cardiac condition (for example heart failure), and the inflammation treatment is provided in addition to the cardiac treatment.

In some embodiments, the indications may occur concomitantly or separately from each other.

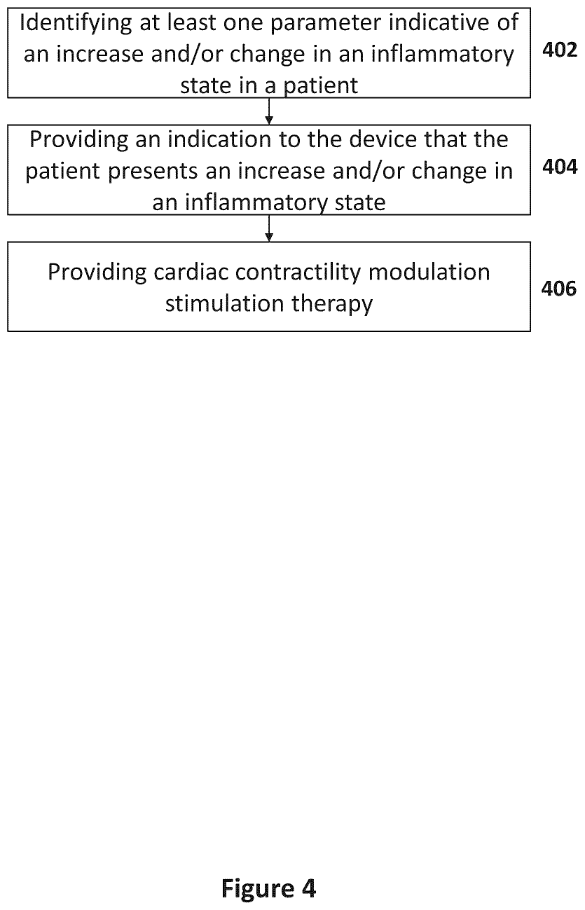

Referring now to FIG. 4, showing a flowchart of an exemplary method to suppress patient cardiac inflammation condition and/or an inflammation condition in a patient presenting a cardiac condition, according to some embodiments of the invention.

In some embodiments, the patient is assessed for one or more parameters indicative of an increase and/or a change in an inflammatory state 402.

In some embodiments, during or after such indication has been identified and it is within the margins of activation for a treatment, the patient and/or physician may trigger cardiac contractility modulation treatment, for example by providing an indication to the implanted device that the event (increase and/or change in inflammation state) is happening 404, for a set period of time from the moment the patient/physician has manually triggered the device 406. For example, such period can be at least 1 min. In some embodiments, such period is typically 5 hours. In some embodiments, such period is more than 5 hours and as needed until a reduction and/or a change in the one or more parameters indicative of an increase and/or a change in inflammatory state have changed. In some embodiments, the reduction and/or a change in the one or more parameters is from about 50% to about 80%, optionally from about 40% to about 90%, optionally from about 30% to about 100%.

In some embodiments, the indication is provided automatically to the implanted device by the one or more sensors. For example, temperature data and/or heart rate data received from the one or more sensors is sent to the controller within the implanted device. In some embodiments, the controller will utilize the temperature data and/or heart rate data received to translate it into the fact that the event is occurring according to predetermined parameters provided to the implanted device.

In some embodiments, the cardiac contractility modulation treatment is triggered, for example, by providing a dedicated external device or magnet that, by turning it on or placing it in the proximity of the implantable device, will activate the cardiac contractility modulation treatment.

In some embodiments, the cardiac contractility modulation stimulation is provided during late night and early morning time, which are the times when inflammation is exacerbated and symptoms and mortality rates are highest.

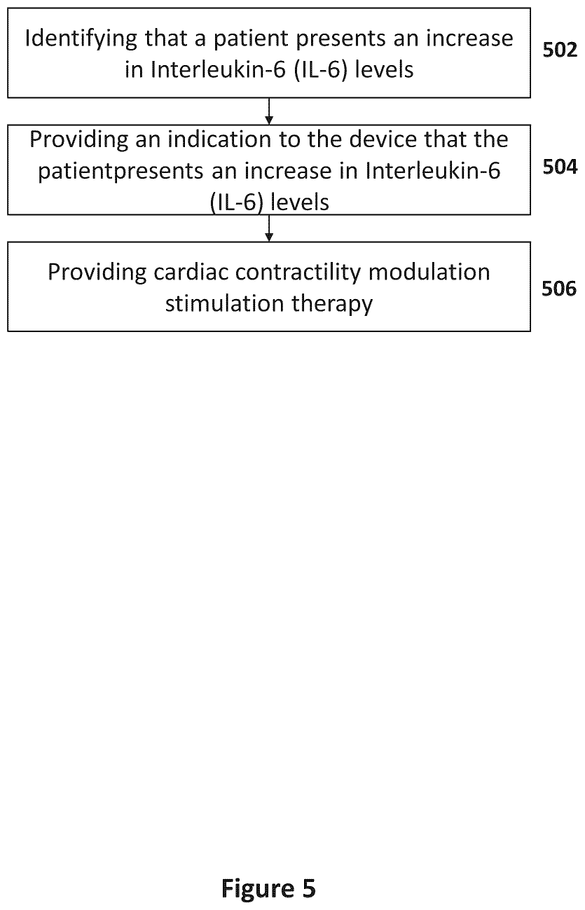

Referring now to FIG. 5, showing a flowchart of an exemplary method to suppress patient cardiac inflammation condition and/or an inflammation condition in a patient presenting a cardiac condition, according to some embodiments of the invention.

In some embodiments, the patient is assessed for levels of Interleukin-6 (IL-6), for example by performing a blood test. In some embodiments, elevated levels of IL-6 are identified in the patient 502.

In some embodiments, during such event, the patient and/or physician may trigger cardiac contractility modulation treatment, for example by providing an indication to the implanted device that the event is happening 504, for a set period of time from the moment the patient/physician has manually triggered the device 506. For example, such period can be at least 1 min. In some embodiments, such period is typically 5 hours. In some embodiments, such period is more than 5 hours and as needed until a reduction in IL-6 levels is achieved. In some embodiments, the reduction in the level of the inflammation markers (IL-6) is from about 50% to about 80%, optionally from about 40% to about 90%, optionally from about 30% to about 100%.

In some embodiments, the cardiac contractility modulation treatment is triggered, for example, by providing a dedicated external device or magnet that, by turning it on or placing it in the proximity of the implantable device, will activate the cardiac contractility modulation treatment.

In some embodiments, the cardiac contractility modulation stimulation is provided during late night and early morning time, which are the times when inflammation is exacerbated and symptoms and mortality rates are highest.

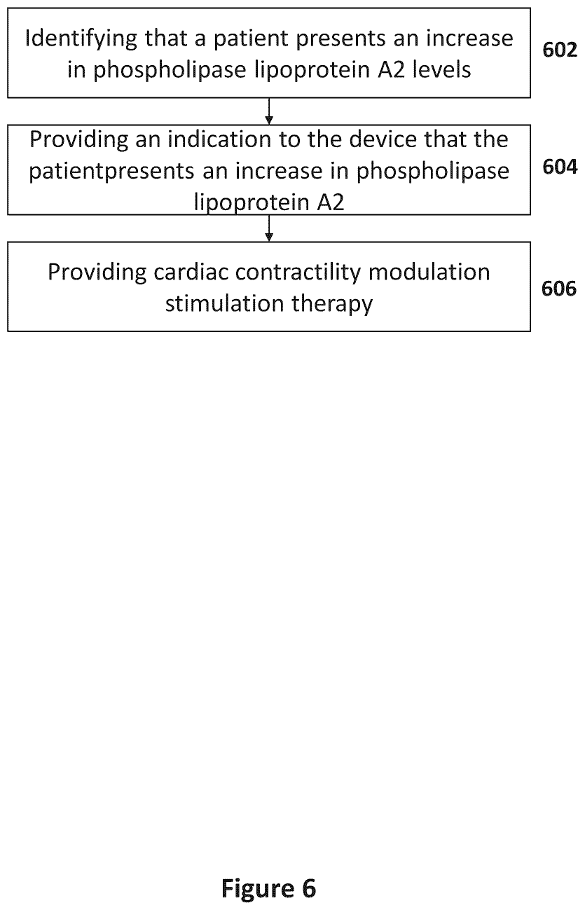

Referring now to FIG. 6, showing a flowchart of an exemplary method to suppress patient cardiac inflammation condition and/or an inflammation condition in a patient presenting a cardiac condition, according to some embodiments of the invention.

In some embodiments, the patient is assessed for levels of phospholipase lipoprotein A2, for example by performing a blood test. In some embodiments, elevated levels of phospholipase lipoprotein A2 are identified in the patient 602.

In some embodiments, during such event, the patient and/or physician may trigger cardiac contractility modulation treatment, for example by providing an indication to the implanted device that the event is happening 604, for a set period of time from the moment the patient/physician has manually triggered the device 606. For example, such period can be at least 1 min. In some embodiments, such period is typically 5 hours. In some embodiments, such period is more than 5 hours and as needed until a reduction in phospholipase lipoprotein A2 levels is achieved. In some embodiments, the reduction in the level of the inflammation markers (phospholipase lipoprotein A2) is from about 50% to about 80%, optionally from about 40% to about 90%, optionally from about 30% to about 100%.

In some embodiments, the cardiac contractility modulation treatment is triggered, for example, by providing a dedicated external device or magnet that, by turning it on or placing it in the proximity of the implantable device, will activate the cardiac contractility modulation treatment.

In some embodiments, the cardiac contractility modulation stimulation is provided during late night and early morning time, which are the times when inflammation is exacerbated and symptoms and mortality rates are highest.

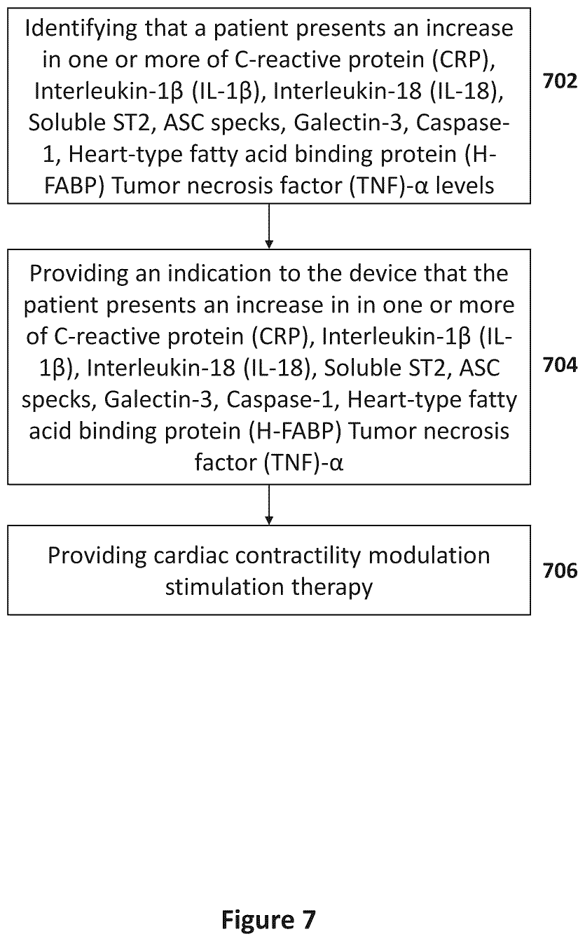

Referring now to FIG. 7, showing a flowchart of an exemplary method to suppress patient cardiac inflammation condition and/or an inflammation condition in a patient presenting a cardiac condition, according to some embodiments of the invention.

In some embodiments, the patient is assessed for levels of phospholipase lipoprotein A2, for example by performing a blood test. In some embodiments, elevated levels of in one or more of C-reactive protein (CRP), Interleukin-1β (IL-1β), Interleukin-18 (IL-18), Soluble ST2, ASC specks, Galectin-3, Caspase-1, Heart-type fatty acid binding protein (H-FABP) Tumor necrosis factor (TNF)-α are identified in the patient 702.

In some embodiments, during such event, the patient and/or physician may trigger cardiac contractility modulation treatment, for example by providing an indication to the implanted device that the event is happening 704, for a set period of time from the moment the patient/physician has manually triggered the device 706. For example, such period can be at least 1 min. In some embodiments, such period is typically 5 hours. In some embodiments, such period is more than 5 hours and as needed until a reduction in one or more of C-reactive protein (CRP), Interleukin-1β (IL-1β), Interleukin-18 (IL-18), Soluble ST2, ASC specks, Galectin-3, Caspase-1, Heart-type fatty acid binding protein (H-FABP) Tumor necrosis factor (TNF)-α levels is achieved. In some embodiments, the reduction in the level of the inflammation markers is from about 50% to about 80%, optionally from about 40% to about 90%, optionally from about 30% to about 100%.

In some embodiments, the cardiac contractility modulation treatment is triggered, for example, by providing a dedicated external device or magnet that, by turning it on or placing it in the proximity of the implantable device, will activate the cardiac contractility modulation treatment.

In some embodiments, the cardiac contractility modulation stimulation is provided during late night and early morning time, which are the times when inflammation is exacerbated and symptoms and mortality rates are highest.

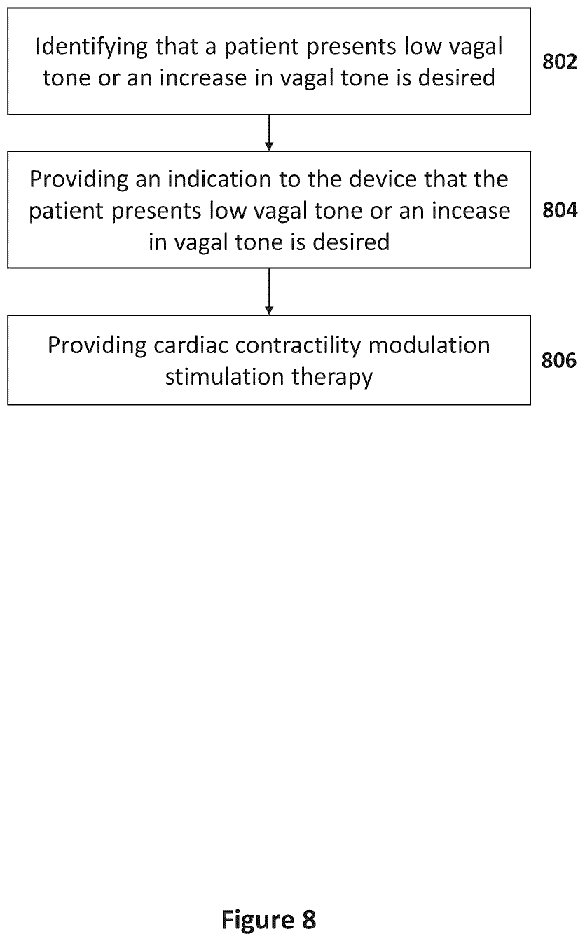

Referring now to FIG. 8, showing a flowchart of an exemplary method to suppress patient cardiac inflammation condition and/or an inflammation condition in a patient presenting a cardiac condition, according to some embodiments of the invention.

In some embodiments, the patient is assessed for low vagal tone activity levels or, in cases where vagal tone levels are normal, an increase of vagal tone activity is desired. In some embodiments, low vagal tone activity levels or, in cases where vagal tone levels are normal, an increase of vagal tone activity is desired are identified in the patient 802.

In some embodiments, during such event, the patient and/or physician may trigger cardiac contractility modulation treatment, for example by providing an indication to the implanted device that the event is happening 804, for a set period of time from the moment the patient/physician has manually triggered the device 806. For example, such period can be at least 1 min. In some embodiments, such period is typically 5 hours. In some embodiments, such period is more than 5 hours and as needed until an increase in vagal tone is achieved. In some embodiments, the increase in the vagal tone activity is from about 50% to about 80%, optionally from about 40% to about 90%, optionally from about 30% to about 100%.

In some embodiments, the indication is provided automatically to the implanted device by the one or more sensors. For example, data received from the one or more heart rate sensors is sent to the controller within the implanted device. In some embodiments, the controller will utilize the data received to translate it into the fact that the event is occurring according to predetermined parameters provided to the implanted device.

In some embodiments, the cardiac contractility modulation treatment is triggered, for example, by providing a dedicated external device or magnet that, by turning it on or placing it in the proximity of the implantable device, will activate the cardiac contractility modulation treatment.

In some embodiments, the cardiac contractility modulation stimulation is provided during late night and/or early morning time, which are the times when inflammation is exacerbated and symptoms and mortality rates are highest.

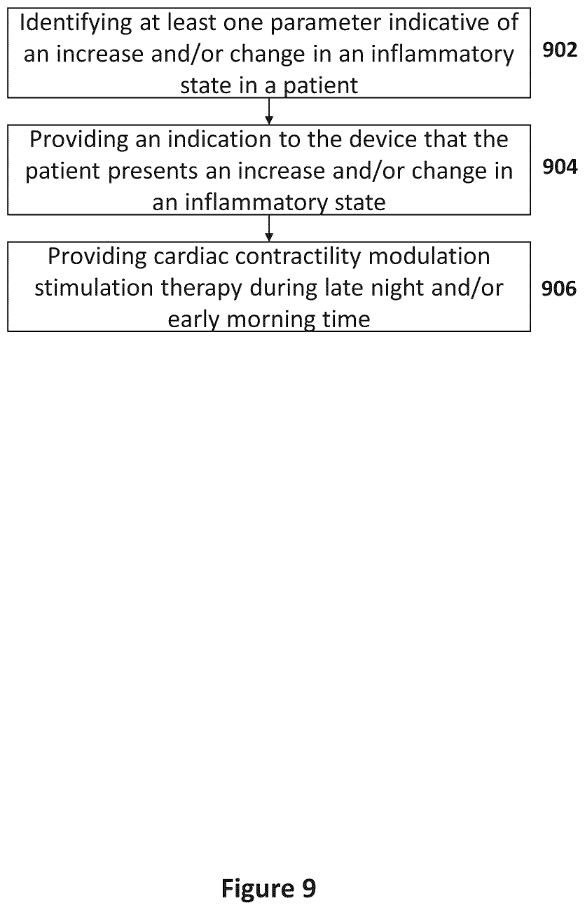

Referring now to FIG. 9, showing a flowchart of an exemplary method to suppress patient cardiac inflammation condition and/or an inflammation condition in a patient presenting a cardiac condition, according to some embodiments of the invention.

In some embodiments, the patient is assessed for one or more parameters indicative of an increase and/or a change in an inflammatory state 902.

In some embodiments, during or after such indication has been identified and it is within the margins of activation for a treatment, the patient and/or physician may trigger cardiac contractility modulation treatment, for example by providing an indication to the implanted device that the event (increase and/or change in inflammation state) is happening 904, for a set period of time and more specifically during late night and/or early morning time 906. For example, such period can be at least 1 min. In some embodiments, such period is typically 5 hours. In some embodiments, such period is more than 5 hours and as needed until a reduction and/or a change in the one or more parameters indicative of an increase and/or a change in inflammatory state have changed. In some embodiments, the reduction and/or a change in the one or more parameters is from about 50% to about 80%, optionally from about 40% to about 90%, optionally from about 30% to about 100%.

In some embodiments, the indication is provided automatically to the implanted device by the one or more sensors. For example, temperature data and/or heart rate data received from the one or more sensors is sent to the controller within the implanted device. In some embodiments, the controller will utilize the temperature data and/or heart rate data received to translate it into the fact that the event is occurring according to predetermined parameters provided to the implanted device.

In some embodiments, the cardiac contractility modulation treatment is triggered, for example, by providing a dedicated external device or magnet that, by turning it on or placing it in the proximity of the implantable device, will activate the cardiac contractility modulation treatment.

In some embodiments, a potential advantage of providing the cardiac contractility modulation stimulation during late night and early morning time, is that those are the times when inflammation is exacerbated and symptoms and mortality rates are highest, which can potentially lead to a reduction in the inflammation in those critical times.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

As used herein, the singular forms “a”, “an” and “the” include plural references unless the context clearly dictates otherwise. For example, the term “a compound” or “at least one compound” may include a plurality of compounds, including mixtures thereof.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A system for suppressing a patient cardiac inflammation condition and/or an inflammation condition in a patient presenting a cardiac condition, comprising an implantable controller, said controller configured to carry out the method of:
a. receiving at least one parameter indicative of an increase and/or a change in an inflammatory state in said patient;
b. providing cardiac contractility modulation stimulation therapy and suppressing a patient cardiac inflammation condition.

2. The system according to claim 1, wherein said at least one parameter is one or more of:
a. a change and/or an increase in one or more inflammatory markers;
b. a change and/or increase in a body temperature of said patient;
c. a change and/or an increase in a heart rate (HR) in said patient;
d. a change and/or a reduction in heart rate variability in said patient;
e. a low vagal tone.

3. The system according to claim 2, wherein said one or more inflammatory markers are markers for one or more of: Interleukin-6 and C-reactive protein (CRP).

4. The system according to claim 2, wherein said one or more inflammatory markers are markers for one or more of: phospholipase lipoprotein A2, Interleukin-1β (IL-1β), Interleukin-18 (IL-18), Soluble ST2, ASC specks, Galectin-3, Caspase-1, Heart-type fatty acid binding protein (H-FABP) and Tumor necrosis factor (TNF)-α levels.

5. The system according to claim 1, wherein said system further comprises at least one sensor configured for detecting said at least one parameter and wherein said at least one parameter is received from said at least one sensor; and
wherein said at least one sensor is located within said system and implanted within a body of said patient and/or outside a body of said patient and wherein said indication is transmitted to said implantable controller.

6. The system according to claim 1, wherein said cardiac contractility modulation stimulation therapy comprises providing a pulse amplitude of 7.5V, a pulse width 5 ms and two bipolar pulses per cardiac cycle.

7. The system according to claim 1, wherein said providing comprises one or more of:
a. providing said effective amount of cardiac contractility modulation stimulation therapy for at least 5 hours of stimulation per day;
b. increasing a total of number of pulses provided per day an increment from about 5% to about 200%;
c. increasing a total of number of pulses provided per hour an increment from about 5% to about 200%;
d. increasing a pulse amplitude of pulses provided an increment from about 5% to about 200%;
e. increasing a pulse width of pulses provided an increment from about 5% to about 200%;
f. increasing a number of pulses provided per cardiac cycle an increment from about 5% to about 200%.

8. The system according to claim 7, wherein said increasing is performed by one or more of:
a. automatically by a cardiac contractility modulation stimulation device;
b. manually by said patient.

9. The system according to claim 1, wherein said controller is further configured to receive an indication of an improvement in said at least one parameter, and further configured for amending said effective amount of cardiac contractility modulation stimulation therapy accordingly.

10. The system according to claim 1, wherein said providing comprises one or more of:
a. providing an increased amount of said effective amount of cardiac contractility modulation stimulation therapy during late night and/or during early morning;
b. providing said effective amount of cardiac contractility modulation stimulation therapy during late night hours and/or during early morning hours.

11. A method for suppressing a patient cardiac inflammation condition with cardiac contractility modulation stimulation therapy and/or an inflammation condition in a patient presenting a cardiac condition, the method comprising:
a. identifying at least one parameter indicative of an increase and/or a change in an inflammatory state in said patient;
b. providing cardiac contractility modulation stimulation therapy and suppressing a patient cardiac inflammation condition.

12. The method according to claim 11, wherein said at least one parameter is one or more of:
a. a change and/or an increase in one or more inflammatory markers;
b. a change and/or increase in a body temperature of said patient;
c. a change and/or an increase in a heart rate (HR) in said patient;
d. a change and/or a reduce in heart rate variability in said patient;
e. a low vagal tone.

13. The method according to claim 12, wherein said one or more inflammatory markers are markers for one or more of: Interleukin-6 and C-reactive protein (CRP).

14. The method according to claim 12, wherein said one or more inflammatory markers are markers for one or more of: phospholipase lipoprotein A2, Interleukin-1β (IL-1β), Interleukin-18 (IL-18), Soluble ST2, ASC specks, Galectin-3, Caspase-1, Heart-type fatty acid binding protein (H-FABP) and Tumor necrosis factor (TNF)-α levels.

15. The method according to claim 11, wherein said identifying is performed by at least one sensor configured for detecting said at least one parameter; and wherein said at least one parameter is received from said at least one sensor; and
wherein said at least one sensor is located within a system and implanted within a body of said patient and/or outside a body of said patient and wherein said indication is transmitted to an implantable controller.

16. The method according to claim 11, wherein said effective amount of cardiac contractility modulation stimulation therapy comprises providing a pulse amplitude of 7.5V, a pulse width 5 mS and two bipolar pulses per cardiac cycle.

17. The method according to claim 11, wherein said providing comprises one or more of:
a. providing said effective amount of cardiac contractility modulation stimulation therapy for at least 5 hours of stimulation per day;
b. increasing a total of number of pulses provided per day an increment from about 5% to about 200%;
c. increasing a total of number of pulses provided per hour an increment from about 5% to about 200%;
d. increasing a pulse amplitude of pulses provided an increment from about 5% to about 200%;
e. increasing a pulse width of pulses provided an increment from about 5% to about 200%;
f. increasing a number of pulses provided per cardiac cycle an increment from about 5% to about 200%.

18. The method according to claim 11, wherein said increasing is performed by one or more of:
a. automatically by a cardiac contractility modulation stimulation device;
b. manually by said patient.

19. The method according to claim 11, further comprising receiving an indication of an improvement in said at least one parameter, and further comprising amending said effective amount of cardiac contractility modulation stimulation therapy accordingly.

20. The method according to claim 11, wherein said providing comprises one or more of:
a. providing an increased amount of said effective amount of cardiac contractility modulation stimulation therapy during late night and/or during early morning; and
b. providing said effective amount of cardiac contractility modulation stimulation therapy during late night hours and/or during early morning hours.
